(19) United States
(12) Patent Application Publication
(10) Pub. No.: US 2025/0228747 A1
(43) Pub. Date: Jul. 17, 2025

(54) NASOJEJUNAL FEEDING TUBE WITH EXPANDABLE WIRE FOR RETENTION

(71) Applicant: BOSTON SCIENTIFIC SCIMED, INC., Maple Grove, MN (US)

(72) Inventors: Nathan Thomas CUMMINGS, Worcester, MA (US); Peter L. DAYTON, Brookline, MA (US); Laura Elizabeth CHRISTAKIS, Framingham, MA (US); John B. GOLDEN, Norton, MA (US); Paul SMITH, Smithfield, RI (US); Ryan V. WALES, Northborough, MA (US); Kapil GUPTA, Boston, MA (US); John Thomas FAVREAU, Charlton, MA (US); Rosangel Aimee RAMOS ESPINOZA, Boston, MA (US); Jeff GRAY, Sudbury, MA (US)

(21) Appl. No.: 19/016,058

(22) Filed: Jan. 10, 2025

Related U.S. Application Data
(60) Provisional application No. 63/621,842, filed on Jan. 17, 2024.

(51) Int. Cl. A61J 15/00

(52) U.S. Cl. CPC A61J 15/0019; A61J 15/0069

(57) ABSTRACT

A device includes a flexible catheter and at least one wirefrom. The catheter includes a first lumen extending therethrough and is sized and shaped to extend to a target location within a gastrointestinal (GI) system. A first distal portion of the catheter includes one or more exit ports communicating with the first lumen for administering a fluid to the location. The wireform is attached around the catheter or deployable from within the catheter, the wireform being formed so that, in a first state, the wireform assumes a first shape suitable for guiding the catheter to the location and, in a second state, the wireform assumes a second shape in which the wire extends away from the catheter in at least one radial direction for contacting a wall of the location to anchor the catheter in the location while administering the fluid via the first lumen.

### PRIORITY CLAIM

The present disclosure claims priority to U.S. Provisional Patent Application Ser. No. 63/621,842 filed Jan. 17, 2024; the disclosure of which is incorporated herewith by

### FIELD

The present disclosure relates to tubes and/or catheters with retention features for anchoring in the small bowel.

### BACKGROUND

Pyloric stenosis constitutes a narrowing of the gastric outlet (pylorus) causing difficulties in passing food from the stomach into the small bowel. This can lead to a gastric outlet obstruction (stricture). The inability to pass food into the small bowel can reduce or completely impede a person's ability to digest food and absorb nutrients. There are several solutions to this problem, including placing a nasojejunal (NJ) feeding tube in the small bowel, e.g., jejunum. Another solution includes the creation of a surgical gastro-jejunostomy bypass bridging the stomach and the jejunum to bypass the area of the stricture. NJ tubes can be used for assisting endoscopic ultrasound guided gastroenterostomy (EUS-GE) procedures by instilling contrast or another fluid in the jejunum. In one example, contrast is provided via the NJ tube to help make a target visible to an operating physician, e.g., for a procedure including piercing the stomach and the jejunum and placing a stent (e.g., an Axios™ stent) therebetween to bypass the pylorus.

One issue with NJ tubes is that due to movements of the patient and/or the digestive organs, the tube may drift out of position so that fluids discharged by the tube are provided to an unintended location (i.e., as the outlet of the tube is no longer located in a target location). In one example, the NJ tube may migrate proximally during use. In feeding tube applications, this proximal drift may cause nutrients to be provided further upstream in the small bowel, or in the stomach proximal to the stricture, which would eliminate the benefits of the tube and may cause issues with digestion. In EUS-GE applications, the drift of the NJ tube can cause fluids (e.g., contrast fluid) to be provided to a location spaced from an intended target area. The stents for bypassing the pylorus may be mis-deployed if the physician is confused or misdirected due to the contrast fluid being provided to an unintended location during gastro-jejunostomy procedures.

### SUMMARY

The present disclosure relates to a device for administering a fluid to a patient anatomy. The device includes a flexible catheter including a first lumen extending therethrough, the catheter being sized and shaped to extend to a target location within a gastrointestinal (GI) system, a first distal portion of the catheter including one or more exit ports communicating with the first lumen for administering a fluid to the target location. In addition, the device includes at least one wireform attached around the catheter or deployable from within the catheter, the wireform being formed so that, in a first state, the wireform assumes a first shape suitable for guiding the catheter to the target location and, in a second state, the wireform assumes a second shape in which the wire extends away from the catheter in at least one radial direction for contacting a wall of the target location to anchor the catheter in the target location while administering the fluid via the first lumen.

In an embodiment, the wireform is a stent comprising a wire mesh with a tubular structure, the stent assuming the second state under a natural bias and being collapsible into the first state around a second distal portion of the catheter, wherein the stent is releasable from the first state to transition into the second state by actuation proximal to the catheter.

In an embodiment, the device further includes a sheath disposed around the second distal portion of the catheter, the sheath being translatable relative to the catheter from a first position to a second position. The stent is constrained by the sheath around the catheter in the first state when the sheath is in the first position and translating the sheath into the second position unconstrains the stent to allow the stent to assume the second state.

In an embodiment, the sheath can be translated from the second position to the first position so that the stent can be repositioned after initial deployment or so that the device can be withdrawn through the GI system.

In an embodiment, the wireform is a coil extending from a first end fixed to an outer surface of the catheter in a helical shape to a second end not fixed to the catheter, the coil assuming the first state when the second end is located away from the first end such that the coil is lengthened and the helical shape is compressed radially, the coil assuming the second state when the second end is drawn toward the first end such that the coil is shortened and the helical shape is expanded radially.

In an embodiment, the device further includes a collar disposed around a second distal portion of the catheter, the second end of the coil fixed to the collar, the collar being translatable relative to the catheter between a first position and a second position. The coil is lengthened by the collar in the first state when the collar is in the first position and translating the collar into the second position provides a compressive force to the coil to transition into the second state.

In an embodiment, the wireform comprises a Y-shape, a waveform, a C-shape, or a coiled mass of wire.

In an embodiment, the wireform in the first state is translatable from a first position where the wireform is within and constrained by an interior of the catheter to a second position where the wireform extends out a distal end of the catheter and assumes the second state.

In an embodiment, the device further includes multiple wires, each wire extending longitudinally from a proximal end to a distal end, the distal end being translatable between a first position and a second position, the multiple wires being spaced circumferentially around the catheter. The wires in the first state are straight and translating the distal ends proximally causes the wires to bend radially outward relative to the catheter.

In an embodiment, the wireform is a spring attached to the catheter on a first end and not attached to the catheter on a second end, the spring assuming the first state when the second end is located away from the first end such that the coil is lengthened and the spring is compressed radially and spaces between rotations of the spring are open, the coil assuming the second state when the second end is drawn toward the first end such that the coil is shortened, the spring is expanded radially, and the spaces between the rotations of the spring are drawn close to grasp adjacent tissue.

In an embodiment, the device further includes at least one further wireform, the wireform comprising a distal anchor and the further wireform comprising a proximal anchor, fluid being instilled between the proximal and distal anchors to create a visible volume of fluid.

In an embodiment, the device further includes a first member extending through the first lumen of the catheter, wherein the wireform is a first flange fixed adjacent to a distal end of the first member, the first member being translatable between a first position wherein the first flange is within and constrained by the first lumen and a second position. The first flange extends out a distal end of the catheter and is allowed to assume the second shape to engage an inner wall of a small bowel.

In an embodiment, the device further includes a second member extending outside the catheter, the second member being translatable between a first position and a second position; and a second flange fixed adjacent to a distal end of the second member. The catheter including a pocket outside the catheter, wherein the second flange is within and constrained by the pocket when the second member is in the first position and the second flange extends out a proximal end of the pocket when the second member is in the second position so that the second flange is allowed to assume the second shape to engage the inner wall of the small bowel at a location proximal to that of the first flange.

In an embodiment, the fluid is instilled through the first lumen out the distal end of the catheter on a proximal side of the first flange so that a visible volume of fluid is created.

In addition, the present disclosure relates to a device for providing a target location for an endoscopic ultrasound guided gastroenterostomy (EUS-GE) procedure. The device includes a flexible catheter including a first lumen extending therethrough, the catheter being sized and shaped to extend to a target location within a gastrointestinal (GI) system; a wire extending from a proximal end to a distal end that, in a first state, is disposed fully within the first lumen; and a pushing element sized and shaped to extend through a proximal end of the first lumen so that a distal end of the pushing element can contact the proximal end of the wire to push the wire out a distal end of the catheter.

In a second state, the wire is pushed out the distal end of the catheter as the catheter is withdrawn through the GI system, the wire being formed so that, in the first state, the wire assumes a first shape constrained by the first lumen suitable for guiding the catheter to the target location and, in the second state, the wire assumes a second shape in which a medial portion of the wire curves to bring the proximal end and the distal end toward each other, the wire being deployed in the GI system so that the distal end contacts a jejunal wall and the proximal end contacts a stomach wall to provide a target under visualization.

In addition, the present disclosure relates to a method for administering a fluid to a patient anatomy. The method includes guiding a flexible catheter to a target location within a gastrointestinal (GI) system, the catheter including a first lumen extending therethrough, a first distal portion of the catheter including one or more exit ports communicating with the first lumen for administering a fluid to the target location; transitioning at least one wireform from a first state to a second state, the at least one wireform being attached around the catheter or deployable from within the catheter, the wireform being formed so that, in the first state, the wireform assumes a first shape suitable for guiding the catheter to the target location and, in the second state, the wireform assumes a second shape in which the wire extends away from the catheter in at least one radial direction for contacting a wall of the target location to anchor the catheter in the target location; and administering the fluid via the first lumen.

In an embodiment, the wireform is a stent comprising a wire mesh with a tubular structure, the stent assuming the second state under a natural bias and being collapsible into the first state around a second distal portion of the catheter, wherein the stent is releasable from the first state to transition into the second state by a sheath disposed around the second distal portion of the catheter, the sheath being translatable relative to the catheter from a first position to a second position, wherein the stent is constrained by the sheath around the catheter in the first state when the sheath is in the first position and translating the sheath into the second position unconstrains the stent to allow the stent to assume the second state.

In an embodiment, the wireform is a coil extending from a first end fixed to an outer surface of the catheter in a helical shape to a second end not fixed to the catheter, the coil assuming the first state when the second end is located away from the first end such that the coil is lengthened and the helical shape is compressed radially, the coil assuming the second state when the second end is drawn toward the first end such that the coil is shortened and the helical shape is expanded radially via a collar disposed around a second distal portion of the catheter, the second end of the coil fixed to the collar, the collar being translatable relative to the catheter between a first position and a second position. The coil is lengthened by the collar in the first state when the collar is in the first position and translating the collar into the second position provides a compressive force to the coil to transition into the second state.

In an embodiment, the wireform comprises a Y-shape, a waveform, a C-shape, or a coiled mass of wire. The wireform in the first state is translatable from a first position where the wireform is within and constrained by an interior of the catheter to a second position where the wireform extends out a distal end of the catheter and assumes the second state.

In an embodiment, the wireform comprises multiple wires, each wire extending longitudinally from a proximal end to a distal end, the distal end being translatable between a first position and a second position, the multiple wires being spaced circumferentially around the catheter. The wires in the first state are straight and translating the distal ends proximally causes the wires to bend radially outward relative to the catheter.

### BRIEF DESCRIPTION

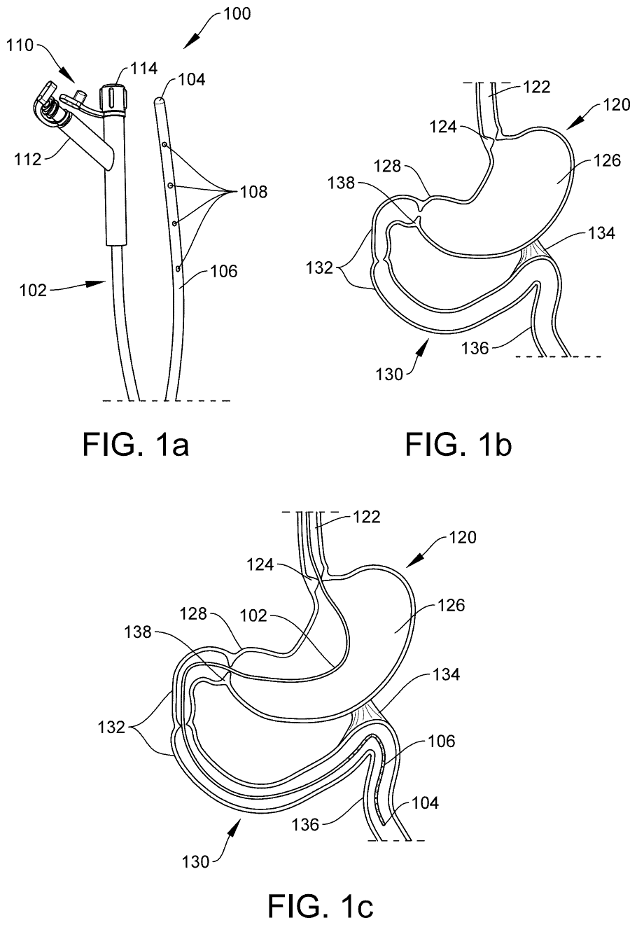

FIG. 1a shows a nasojejunal (NJ) tube device according to one example.

FIG. 1b shows a patient anatomy prior to inserting a NJ tube according to one example.

FIG. 1c shows the catheter of the NJ tube device of FIG. 1a placed in the patient anatomy of FIG. 1b according to one example.

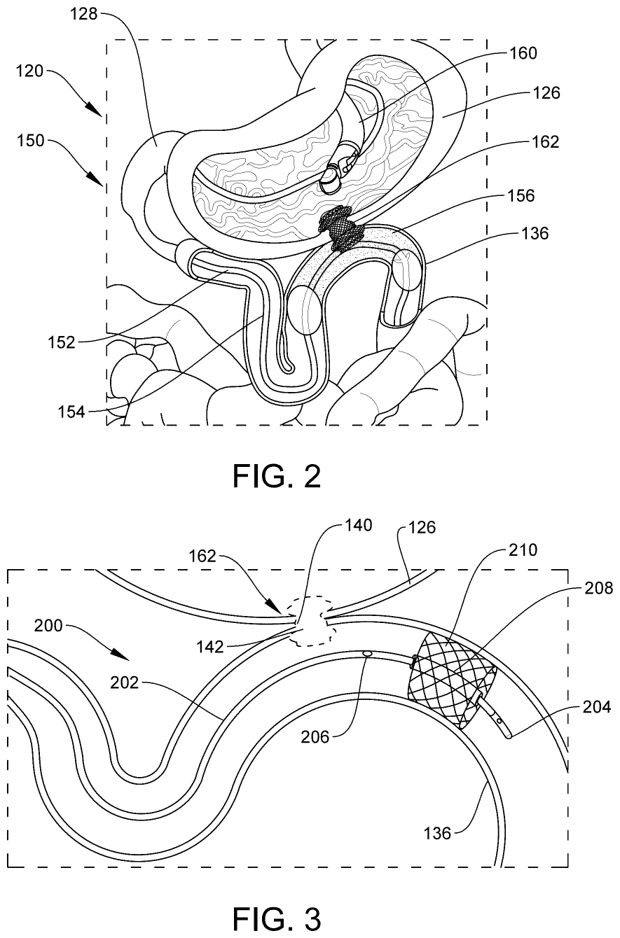

FIG. 2 shows a EUS-GE system for placing a gastro-jejunal stent in the anatomy of FIG. 1b according to one example.

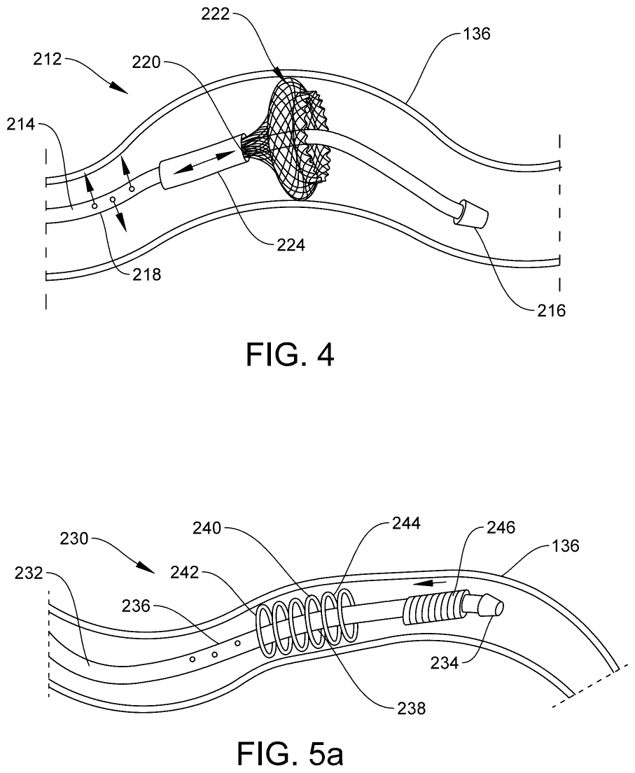

FIG. 3 shows a catheter device including a catheter with a stent fixed thereto in an expanded shape (second shape) within the patient anatomy of FIG. 1b according to a first example of various exemplary embodiments.

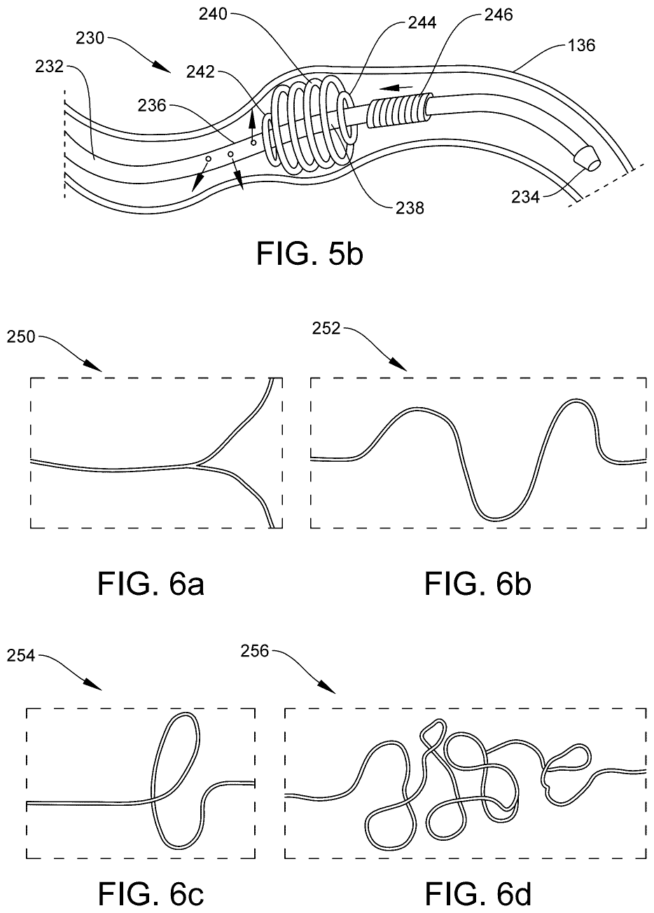

FIG. 4 shows a catheter device including a catheter and a stent fixed thereto in a partially expanded shape (second shape) within the patient anatomy of FIG. 1b, the stent being re-constrainable by a sheath, according to a second example of various exemplary embodiments.

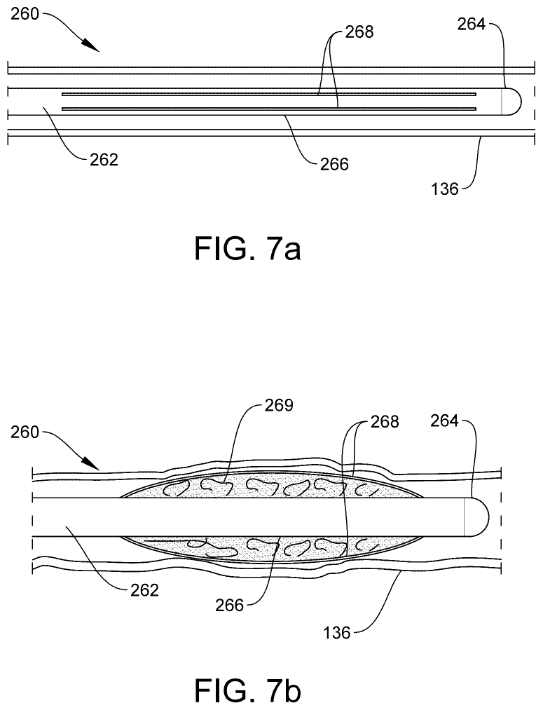

FIG. 5a shows a catheter device including a catheter with a coil fixed thereto in an insertion state (first shape) within the patient anatomy of FIG. 1b according to a third example of these exemplary embodiments.

FIG. 5b shows the catheter device of FIG. 5a with the coil transitioned into an expanded state (second shape) by a collar according to the third exemplary embodiment.

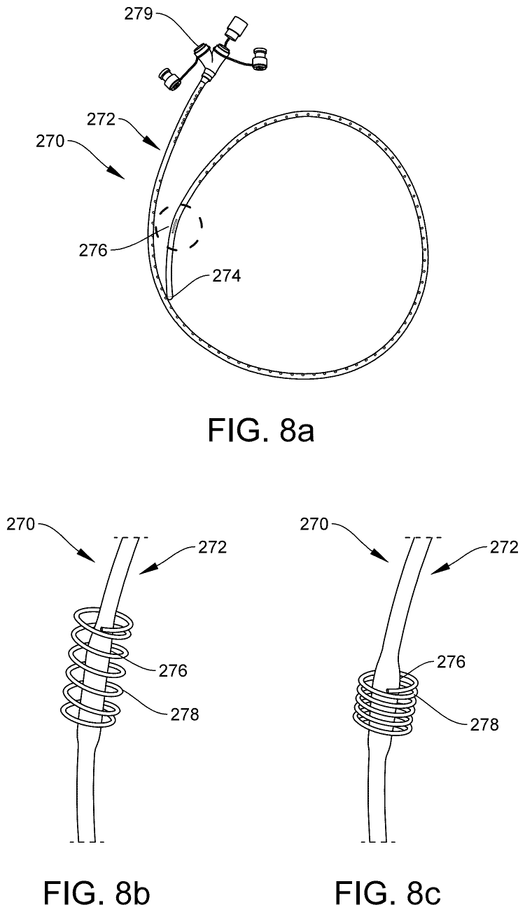

FIG. 6a shows a first wireform according to a fourth example of these exemplary embodiments.

FIG. 6b shows a second wireform according to a fifth example of these exemplary embodiments.

FIG. 6c shows a third wireform according to a sixth example of these exemplary embodiments.

FIG. 6d shows a fourth wireform according to a seventh example of these exemplary embodiments.

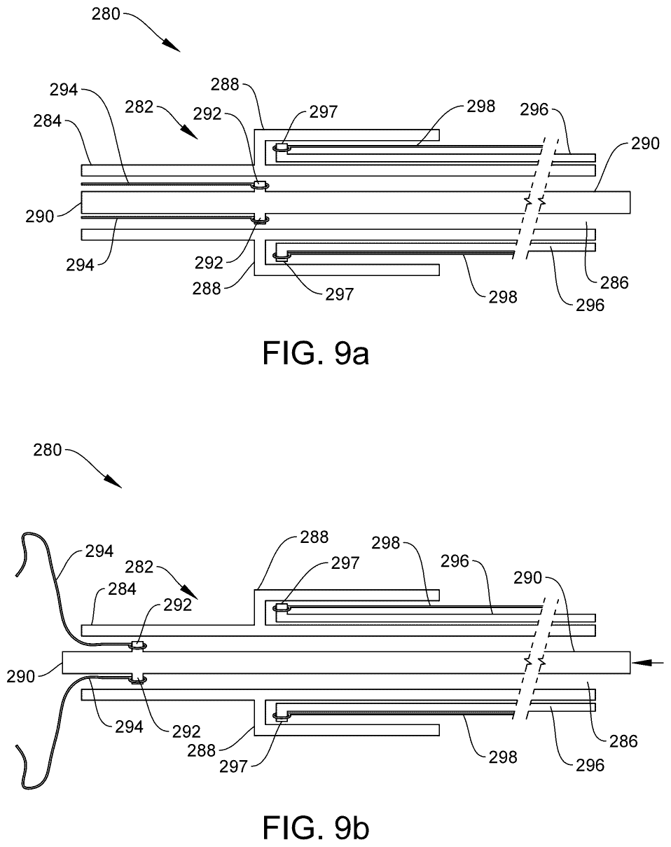

FIG. 7a shows a catheter device including a catheter with multiple longitudinal wires arrayed circumferentially around a distal portion thereof in an insertion state (first shape) within the patient anatomy of FIG. 1b according to an eighth example of these exemplary embodiments.

FIG. 7b shows the catheter device of FIG. 7a with the wires transitioned into an expanded state (second shape) according to the eighth exemplary embodiment.

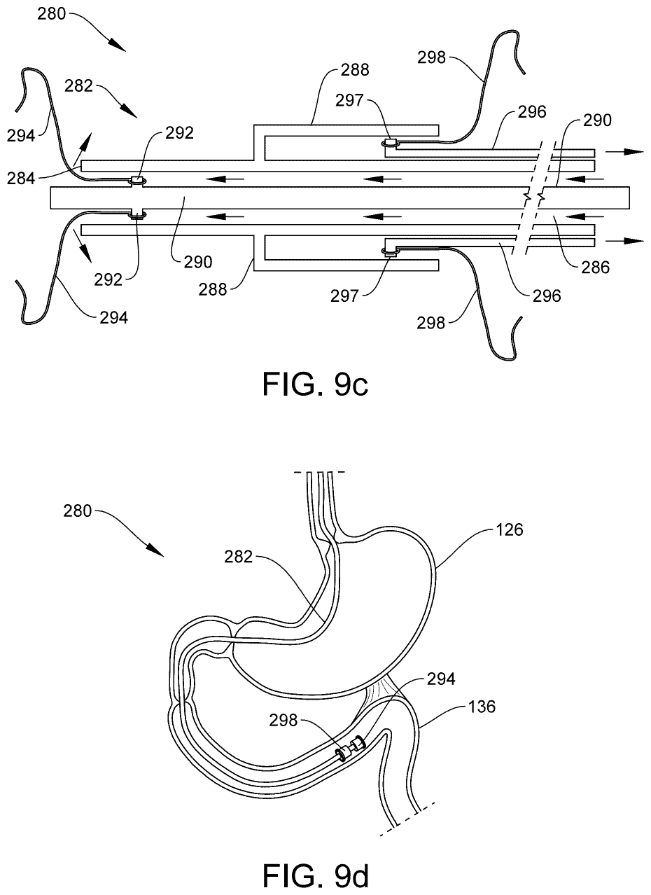

FIG. 8a shows a catheter device including a catheter with a distal portion on which a spring can be fixed according to a ninth example of these exemplary embodiments.

FIG. 8b shows the spring of the catheter device of FIG. 8a in an insertion state (first shape) according to the ninth exemplary embodiment.

FIG. 8c shows the spring of FIG. 8b in an expanded state (second shape) according to the ninth exemplary embodiment.

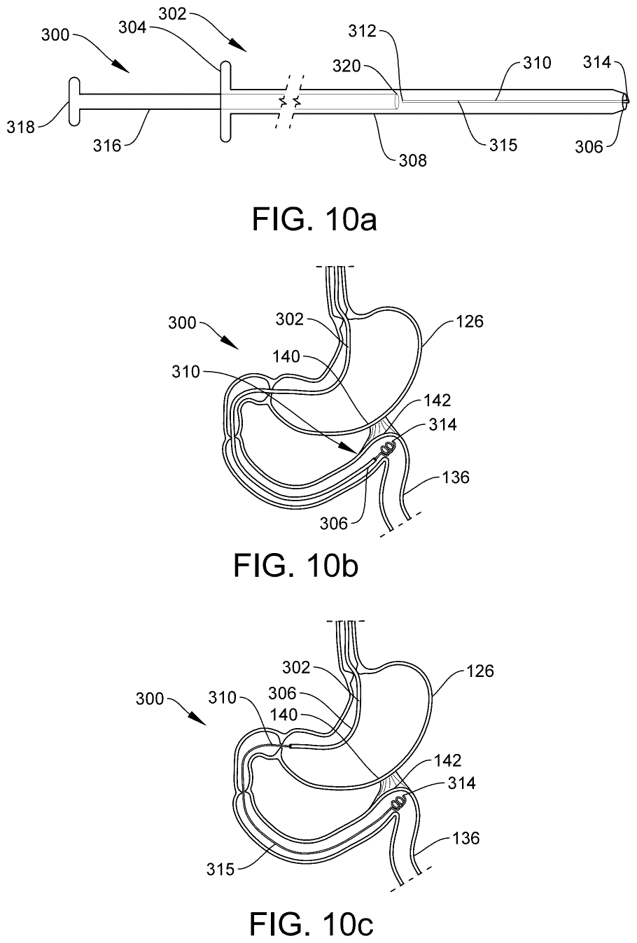

FIG. 9a shows a catheter device including a catheter with a distal flange and a proximal flange in an insertion state (first shape) according to a tenth example of these exemplary embodiments.

FIG. 9b shows the catheter device of FIG. 9a with the distal flange transitioned into an expanded state (second shape) according to the tenth exemplary embodiment.

FIG. 9c shows the catheter device of FIG. 9b with the proximal flange transitioned into an expanded state (second shape) according to the tenth exemplary embodiment.

FIG. 9d shows the catheter device of FIG. 9c deployed in the patient anatomy of FIG. 1b according to the tenth exemplary embodiment.

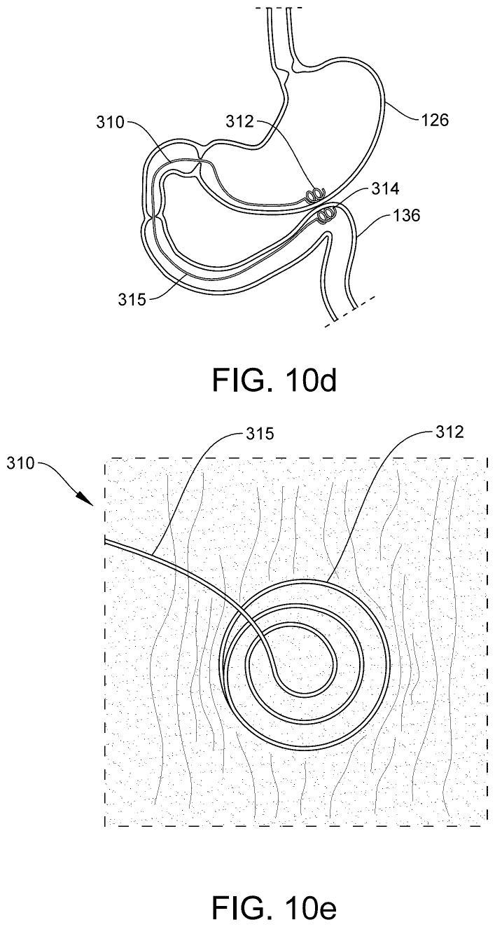

FIG. 10a shows a catheter device including a catheter with a lumen carrying a wire in an insertion state (first shape) according to an eleventh example of these exemplary embodiments.

FIG. 10b shows the catheter device of FIG. 10a deployed in the patient anatomy of FIG. 1b with a distal end of the wire pushed out a distal end of the catheter according to the eleventh exemplary embodiment.

FIG. 10c shows the catheter device of FIG. 10b deployed in the patient anatomy of FIG. 1b with a medial portion of the wire pushed out the distal end of the catheter according to the eleventh exemplary embodiment.

FIG. 10d shows the wire of the catheter device of FIG. 10c in a deployed state (second shape) in the patient anatomy of FIG. 1b according to the eleventh exemplary embodiment.

FIG. 10e shows an alternative view of the wire of the catheter device of FIG. 10d in the deployed state (second shape) in the patient anatomy of FIG. 1b according to the eleventh exemplary embodiment.

### DETAILED DESCRIPTION

The present disclosure may be further understood with reference to the following description and the appended drawings, wherein like elements are referred to with the same reference numerals. The present disclosure relates to nasojejunal (NJ) tube and/or catheter devices including features for retention at a target location in the gastrointestinal (GI) tract. In particular, the exemplary catheter devices include at least one wire or wireform fixed or otherwise coupled to a catheter, the wireform configured to transition from a first shape or state suitable for advancing the catheter through the GI tract to a second shape or state in which the wireform expands in two or three dimensions to engage an inner wall of the small bowel, e.g., the duodenum or the jejunum.

Pyloric stenosis involves a narrowing of the gastric outlet (pylorus) which may cause difficulties in passing food from the stomach to the small bowel. This may lead to gastric outlet obstruction (stricture) reducing or completely impeding the ability of the patient to digest food and absorb nutrients. As indicated above, there are several currently employed treatments for this problem, including placing a nasojejunal (NJ) feeding tube in the small bowel. The NJ tube is fed down the esophagus into the stomach, e.g., via endoscope or guidewire, and guided through the stricture so that nutrients may be provided to the small bowel, e.g., the jejunum, using the tube to bypass the stricture. The NJ tube typically comprises holes along a length of a portion of the tube near the distal end of the tube so that this portion, when positioned as desired, discharges nutrients from the tube into the target anatomical structure (e.g., small bowel) via the holes.

FIG. 1a shows a nasojejunal (NJ) tube device 100 according to one example. The device includes a catheter 102 (e.g., tube) extending from a proximal end (not shown) to a distal end 104 including, for example, an atraumatic distal tip. The catheter 102 is configured to be advanced through the gastrointestinal (GI) tract to a target site for the delivery of fluids (e.g., nutrition, therapeutic substances, contrast, etc.) thereto. The catheter 102 of this example comprises a single lumen or channel although, those skilled in the art will understand that a multi-lumen catheter may also be formed according to the teachings of the disclosed embodiments. The catheter 102 has a distal portion 106 extending proximally from a location near or at the distal end 104 comprising holes 108 configured to discharge a fluid administered through the lumen of the catheter 102 to a target anatomical structure. In short, the distal portion 106 of the catheter 102 extends along a portion of the catheter 102 that is configured to be positioned within a target anatomical area into which fluids are to be supplied via the catheter. The catheter 102 of this embodiment is configured to be guided through the GI tract to a position in which the distal portion 106 is located in the small bowel, e.g., the jejunum.

The NJ tube device 100 of this embodiment further comprises a handle 110 including a port 112 in fluid communication with and configured to access the lumen of the catheter, e.g., to administer the fluid from the port 112 to the target anatomical structure via the lumen of the catheter 102. The catheter 102 is configured (sized and shaped) to be advanced longitudinally through a channel of the handle 110 as the catheter 102 is guided to the target location. The proximal end of the catheter 102 is fixed to a cap 114 configured to interface with the handle 110 when the catheter 102 is fully extended distally relative to the handle 110.

FIG. 1b shows a portion of the patient anatomy 120 prior to inserting a NJ tube according to one example. The patient anatomy 120 comprises a portion of the GI tract including the esophagus 122, the stomach 126 and the small bowel 130 (small intestine). The small bowel 130 includes the duodenum 132, the jejunum 136, and the ileum (not shown). As would be understood by those skilled in the art, the esophageal sphincter 124 corresponds to the end of the esophagus 122 and the start of the stomach 126, the pylorus 128 corresponds to the end of the stomach 126 and the start of the duodenum 132 and the ligament of Treitz 134 is a ligament supporting the small bowel and corresponds to the end of the duodenum 132 and the start of the jejunum 136. Pyloric stenosis refers to a narrowing of the pylorus 128 that can create a stricture 138 preventing or making difficult the passage of food from the stomach 126 to the small bowel 130.

FIG. 1c shows the catheter 102 of the NJ tube device 100 of FIG. 1a placed in the patient anatomy 120 of FIG. 1b. The catheter 102 of the NJ tube device 100 is configured to be passed down the esophagus 122, through the stomach 126, past the stricture 138 (e.g., the pylorus 128 which is narrowed) and into the small bowel 130 under visualization (e.g., via endoscope) by using a guidewire, and/or any other known methods while the handle 110 and a proximal end of the catheter 102 remain outside the patient's body accessible to a user. The catheter 102 is configured (e.g., sized and shaped) to be passed through the duodenum 132 past the ligament of Treitz 134 to extend into the jejunum 136 so that the distal portion 106 comprising the holes 108 is positioned at least partially in the jejunum 136. Once the catheter 102 has been placed at the desired location, fluids can be administered via the handle 110 and discharged into the jejunum 136 via the holes 108.

In this example, the NJ tube device 100 is used for feeding purposes with the fluids administered by the tube comprising nutrients for the patient. In other examples, the fluid can comprise contrast or other therapeutic fluids, e.g., medicines, fluids configured to enhance visualization for assisting endoscopic ultrasound guided gastroenterostomy (EUS-GE) procedures, etc.

Another method for treating a patient with pyloric stenosis includes the creation of a surgical gastro-jejunostomy which forms a bypass directly bridging the stomach and the jejunum to bypass the area of the stricture. NJ tubes can be used to assist with EUS-GE procedures by instilling contrast or another fluid into the jejunum to enhance visualization of the target anatomy. In one example, contrast is provided to show more clearly a target to an operating physician, e.g., for a procedure including piercing the stomach and the jejunum and placing a stent (e.g., an Axios stent) therebetween to bypass the pylorus.

FIG. 2 shows an EUS-GE system 150 for placing a gastro-jejunal stent 162 in the patient anatomy 120 of FIG. 1b according to one example. The EUS-GE system 150 comprises a NJ tube device 152 including a catheter 154 that is configured to be passed into the jejunum 136 so that fluid can be discharged into the jejunum 136. In this example, the fluid comprises contrast fluid 156 to help the operating physician locate the target location.

The EUS-GE system 150 of this example further comprises an endoscope 160 for deploying the stent 162. A piercing element (not shown) is deployed via the endoscope 160 to pierce the stomach 126 and the jejunum 136 as would be understood by those skilled in the art. The stent 162 is placed to bridge the stomach 126 and the jejunum 136 bypassing the pylorus 128.

One issue with NJ tubes is that the tube may drift out of position during use such that fluids discharged by the tube may be provided to an unintended location. In one example, the NJ tube migrates proximally during use. In feeding tube applications, this proximal drift can cause nutrients to be provided further up in the small bowel, or even in the stomach proximal to the stricture, which would cause issues with digestion.

In EUS-GE applications, the drift of the NJ tube can cause fluids (e.g., contrast fluid) to be instilled away from an intended target area. With regard to the gastro-jejunostomy procedure described in FIG. 2, without proper visualization, stents for bypassing the pylorus may be misdeployed. In some examples, the stent may be deployed in a hole created in the stomach but without capturing the jejunum (which may or may not have been pierced by the piercing element); similarly, the stent can be deployed in the hole created in the jejunum but without capturing the stomach; or the stent may be deployed to bridge the stomach and the colon. Accordingly, there is a need for improved devices and mechanisms to retain a tube in a desired location in the GI tract.

According to various exemplary embodiments, systems, devices and methods are described for retaining a catheter, e.g., a nasojejunal tube, at a desired location in the small bowel. The catheter can be retained at a position in the small bowel such that the holes for discharging a fluid are positioned in a desired location, e.g., the jejunum. Some of the devices described herein include a catheter intended for use as a feeding tube, e.g., for extended use (e.g., weeks, months, etc.), and some of the devices described herein include catheters intended for providing other fluids during EUS-GE procedures, as will be described below. However, it is noted that many of the described embodiments are equally applicable for either purpose, as described in detail below.

The terms “NJ tube” and/or “catheter” are used herein to describe a tube configured for deployment within the GI tract for administering a fluid thereto, e.g., for feeding, a therapeutic fluid or a fluid applied to facilitate a procedure such as EUS-GE. The catheters according to the present embodiments generally have a single lumen or channel for passing a fluid through the catheter from a proximal end to a distal portion from which the fluid can be discharged through holes in the wall of the catheter. In some cases, the catheter may not have holes in its wall but rather may have an open distal tip for discharging the fluid. Alternatively, a catheter may have both a series of holes located along a length of a distal portion thereof and an open distal end. In some embodiments, the catheter can include multiple lumens for facilitating the actuation of the retention features, to be described below.

Those skilled in the art understand that a simple catheter comprises a length of tube, e.g., formed of a material such as a plastic and/or polymer (e.g., PVC, PTFE, etc.) with a desired flexibility and/or rigidity, that may assume a substantially straight (longitudinal) or slightly curved shape when no external forces are applied at any location along the length of the tube. However, the simple catheter is typically sufficiently flexible such that during deployment the catheter can bend or curve under external forces imposed by, e.g., an operating physician at the proximal end of the catheter; by contacting an inner wall of an endoscope channel; by contacting inner walls of organs along the GI tract; or by passing the catheter over a guidewire.

Accordingly, the simple catheter can assume many variations of three-dimensional shapes due to its flexibility and various external forces imposed at along its length allowing the catheter to extend along a tortuous path (e.g., along a natural body lumen such as any portion of the GI tract). However, those skilled in the art understand that the ability of the catheter to curve or bend is generally limited and such bending typically proceeds in a snake-like manner, e.g., as body lumens are traversed. Those skilled in the art will ascertain that a simple catheter generally does not expand in shape, for example, radially, nor have additional features for expansion relative to its circular cross-section along its length.

In various exemplary embodiments described herein, a catheter device includes a wire or wireform that can be deployed from a catheter. The wireform may be, for example, coupled to an exterior of a catheter for deployment or may be deployable from within a catheter. The wireform according to these exemplary embodiments is deformable and can transition between a first state or shape (e.g., insertion shape/state or contracted shape/state) into a second state or shape (e.g., deployment shape/state or expanded shape/state). The wireform in the second state can be sized, shaped and otherwise designed to contact an inner wall of a lumen within which the catheter is to reside (e.g., the small bowel) for retaining the catheter therein. Various types of wireform can be used, including but not limited to: wire meshes or stents; springs or coils; various two-dimensional or three-dimensional shapes (e.g., Y shape, wave, etc.); flanges; and other types, to be described in detail below. It should be understood that the term “wire” or “wireform” as used herein may refer to many different configurations or shapes of wire and, in some embodiments, may refer to multiple wires. For example, in some embodiments, the wireform may comprise a number of wires braided into a wire mesh.

In some aspects of these exemplary embodiments, the wireform is fixed to the catheter at one or more locations. From the fix location(s), a remainder of the wireform is permitted to expand relative to the catheter so that, when the wireform is expanded at a suitable location within the target organ (e.g., the small bowel), the wireform engages an inner surface of the small bowel. In other aspects, the wireform can be separated from the catheter and can be advanced through the main lumen or a secondary lumen of the catheter for deployment out of an open distal end of the catheter.

In some aspects, the wireform is configured so that its natural shape, e.g., its shape under no external forces and/or under a natural bias, has at least a portion that is expanded (or assumes a greater two-dimensional area and/or three-dimensional volume) relative to the catheter (e.g., a cross-section thereof) carrying the wireform. In these embodiments, the wireform can be constrained so that it assumes a shape that is contracted, e.g., on or around the exterior of the catheter or within a lumen of the catheter, such that the catheter can be guided through the GI tract to a target location, at which time the wireform can be released so that it deploys into an expanded state to engage the interior of the small bowel. In some aspects, the wireform can be re-constrainable into the contracted shape, e.g., to disengage the wireform from the small bowel for repositioning or to withdraw the catheter.

In other aspects, the wireform is configured so that its natural shape is the contracted shape. In these embodiments, the wireform can be expanded by a further mechanism configured with the catheter, e.g., a collar or other expansion mechanism, or by some internal activation feature, e.g., heat activation of a shape memory wire, to be described in detail below. In still further aspects, the catheter can include multiple wireforms.

In some embodiments, the wireform can be deployed/expanded only once (one-time deployment) while, in other embodiments, the wireform can be transitioned between the contracted shape and the expanded shape multiple times, e.g., re-constrainable.

In the present embodiments, the wireform assumes a first shape suitable for guiding the catheter through the GI tract. The first shape may be referred to herein as a first state, an insertion state, a contracted shape, etc. Those skilled in the art understand that the wireform can assume the first shape or low-profile state to facilitate the guiding of the catheter through a body lumen to a target location in a typical manner, e.g., without excessive contact with the inner walls of the GI tract that might hinder the movement of the catheter through the GI tract. The wireform can be disposed outside the catheter with a minimal profile or can be disposed within the catheter in the first state.

When the wireform is deployed, e.g., unconstrained, at least a portion of the wireform is then transitioned into a second shape. The second shape can include some aspect of expansion, e.g., radial expansion, relative to the contracted shape such that the size of the wireform is increased, e.g., in two or three dimensions, to be described below. The second shape may be referred to herein as an expanded shape, a second state, a deployment state, etc. Those skilled in the art will understand that the wire itself does not need to change diameter or dimensions. Rather, the wire is transitioned to a state in which the wire bends through a complex shape so that this shape defined by the wire is expanded relative to a size of the catheter. For example, a wire that transitions from a straight line to extend along a helix can be said to define a cylinder having a radius that is greater than the radius of the catheter or the wire alone. The radius of space taken up by the wire in this example is equal to the radius of the cylinder defined by the helix along which the wire extends.

In some aspects of these exemplary embodiments, the wireform comprises a stent or a stent-like feature (referred to herein as a “stent” for ease of description). Those skilled in the art understand that the term “stent” generally refers to a structure comprising interwoven or braided wires to form a wire mesh. In its natural (unconstrained) state, the stent can comprise a tube-like shape having a size (e.g., length and cross-sectional diameter) selected for placement within a body lumen. This structure can be compressible and/or constrainable for delivery to a target region using a catheter, at which time it can be expanded and/or unconstrained to transition into its natural size to engage the inner walls of the lumen. Stents are often formed of or include biocompatible metals such as stainless steel, cobalt-chromium alloys, or nitinol. Stents may also be formed of or include (e.g., as a coating) biocompatible polymers. Stents may be uncovered or covered, e.g., to create a barrier to prevent fluid from flowing through the walls of the stent.

In these embodiments, a catheter device includes a catheter and a stent-like expandable feature integral to the catheter providing an anchor for retaining the catheter in the small bowel. The stent prevents migration of the catheter without adding traction to the jejunum lumen. All the materials of these catheter devices may be biocompatible to prevent any irritation or foreign body reaction. In some embodiments, the stent can be formed with a braided wire similar to other conventional stents. The catheter can be anchored in position in the jejunum with an expandable stent near the distal end of the catheter which takes up a larger volume in the jejunum.

In these embodiments, the stent can be fixed to the catheter at either one or both of its proximal end and its distal end. For example, the stent may comprise a proximal and/or distal portion that is shaped as a transitionary region, e.g., having one end attached around the exterior of the catheter (e.g., fixed around its circumference so that this end of the stent does not change shape) and another end that, when the stent is expanded, extends radially or substantially radially outward so that the main (tube-like) body of the stent can extend longitudinally therefrom. The stent can be constrained and/or released via various different mechanisms known to those skilled in the art, e.g., a sleeve, a sheath, a pull wire, etc.

FIG. 3 shows a catheter device 200 including a catheter 202 with a stent 210 fixed thereto in an expanded shape (second shape) within the patient anatomy 120 of FIG. 1b according to a first example of various exemplary embodiments. The patient anatomy 120 comprises portions of the GI tract including the stomach 126 and the small bowel 130, in particular, the jejunum 136, similar to the preceding figures. In this exemplary procedure, the catheter device 200 is introduced in the esophagus, passed through the stomach 126 past the stricture and placed such that a distal end 204 thereof is within the small bowel 130.

The catheter 202 extends from a proximal end (not shown) to a distal end 204 with an atraumatic distal tip. In this example, a first distal portion (not labeled) of the catheter 202 includes a single hole 206 for providing an outlet for fluid introduced via the lumen of the catheter 202. In other examples, the first distal portion can comprise multiple holes. A second distal portion 208 of the catheter 202 provides a surface over which the stent 210 can be constrained in the first state.

The stent 210 can be fixed to the catheter 202 in various ways, e.g., at the proximal end and/or at the distal end of the stent 210. The stent 210 can be maintained in the first shape (insertion state) in various ways, e.g., using a sheath or sleeve, as would be understood by those skilled in the art. To transition the stent 210 into the second shape, various mechanisms can be used depending on the means by which the stent 210 is fixed/constrained to the catheter 202, e.g., by pull wire. In this example, once the stent 210 has expanded, it remains in the second shape for the remainder of its deployment. That is, the stent 210 is not re-constrainable (able to be transitioned back into the first shape), thus, once deployed, it remains in fixed location within the small bowel 130. The stent 210 can be removed at a later time using a different device, e.g., in an endoscopic procedure.

In this example, the second distal portion 208 of the catheter 202 carrying the stent 210 is distal to the first distal portion comprising the hole 206 for discharging the fluid. The location of the second distal portion 208 relative to the first distal portion including the hole 206 may depend on a desired location for discharging the fluid relative to a desired location for contacting the small bowel to retain the position of the catheter 202.

In this example, the desired location for discharging the fluid is within the jejunum 136, e.g., for assisting an EUS-GE procedure such as that discussed with regard to FIG. 2. Accordingly, the catheter 202 can be passed through the small bowel 130 so that the first distal portion is at or adjacent to a desired location 142 in the jejunum for placing the stent 162. The stent 210 can be deployed from the second distal portion 208 to provide an anchor location distal to the desired location 142 in the jejunum. It should be understood that, in other embodiments, the second distal portion 208 and stent 210 can be located differently on the catheter 202 relative to the first distal portion comprising the hole(s) 206. For example, the second distal portion 208 can be located proximally to the first distal portion such that the anchor location is proximal to the hole(s) 206 for discharging the fluid, or the first distal portion and second distal portion 208 can coincide wholly or in part.

In this example, the catheter device 200 is for assisting the EUS-GE procedure and the fluid comprises contrast fluid for visualizing the small bowel 130. The fluid can be provided in the small bowel 130 such that a piercing element deployed from an endoscope can penetrate the stomach 126 at a desired location 140 and further penetrate the jejunum 136 at the desired location 142 so that the stent 162 can be placed to bridge the desired locations 140, 142. However, it should be understood that a similar catheter device may be deployed so that the first distal portion and/or the second distal portion 208 is placed further proximally, e.g., in the duodenum or toward the beginning of the jejunum, so that a fluid comprising nutrients can be provided at a location where the nutrients will be digested at a proper location.

In another aspect of these exemplary embodiments, a catheter device can include a stent that is re-constrainable for removal and/or repositioning. In one embodiment, the catheter device can include a sheath disposed around the catheter that deploys the stent when retracted proximally and can re-constrain the stent when the sheath is moved distally. The sheath can be translated proximally and distally in various ways, including, e.g., by pull wire.

FIG. 4 shows a catheter device 212 including a catheter 214 with a stent 222 fixed thereto in a partially expanded shape (second shape) within the patient anatomy 120 of FIG. 1b, the stent 222 being re-constrainable by a sheath 224, according to a second example of various exemplary embodiments. Similar to the catheter device 200 of FIG. 3, the catheter device 212 can be introduced in the esophagus, passed through the stomach past the stricture and placed such that a distal end 216 thereof is within the small bowel. The patient anatomy 120 shown in FIG. 4 comprises the jejunum 136, similar to the preceding figures.

The catheter 214 extends from a proximal end (not shown) to a distal end 216 with an atraumatic distal tip. In this example, a first distal portion 218 of the catheter 214 includes multiple holes (not labeled) for providing an outlet for fluid introduced via the lumen of the catheter. In other examples, the first distal portion 218 can comprise a single hole. In this example, the catheter device 212 further includes the stent 222 and the sheath 224. A second distal portion 220 of the catheter 214 provides a surface over which the stent 222 can be constrained in the first state via the sheath 224. In this example, the second distal portion 220 is distal to the first distal portion 218 comprising the holes. In other embodiments, the second distal portion 220 over which the stent 222 and the sheath 224 are disposed can be proximal to the first distal portion 218 or coincide wholly or in part with first distal portion 218.

The stent 222 can be fixed to the catheter 214 in various ways, similar to the stent 162 of the catheter device 200 of FIG. 3, e.g., at one or both of its proximal and distal ends. The stent 222 can be maintained in the first shape (insertion state) by the sheath 224 when the sheath 224 is positioned around the stent 222 on the second distal portion 220 of the catheter 214, e.g., in a first position. In this example, to transition the stent 222 into the second shape (expanded state), the sheath 224 can be withdrawn proximally relative to the catheter 214 to a second position (or to any position between the first position (distal-most) and second position (proximal-most)).

The stent 222 can expand to varying degrees (e.g., the second shape may vary in length and radial expansion) based on the position of the sheath 224 and the degree to which the sheath 224 uncovers the stent 222. To transition the stent 222 back into the first shape, the sheath 224 can be advanced distally relative to the catheter 214. The sheath 224 can be translated proximally and distally relative to the catheter 214 via any suitable mechanism, e.g., pull wire. It should be understood that the stent and sheath described above may be configured differently so that, e.g., distal advancement of the sheath 224 uncovers the stent 222. The stent 222 can be constrained in the first state to withdraw the catheter device 212 from the patient anatomy.

Similar to the catheter device 200 described above with regard to FIG. 3, the catheter device 212 can be used for providing fluid for visualizing the small bowel for an EUS-GE procedure or can be placed further proximally for use as a feeding tube.

In another embodiment, a catheter device includes an expandable coil wound around an outside surface of the catheter near its distal end. The catheter device further includes a collar disposed around the outside surface of the catheter. The coil is fixed to the catheter at its proximal end and fixed to the collar at its distal end while the remainder of the coil is free to float around the OD of the catheter. The coil therefore can be stretched or compressed depending on the position of the collar. The collar is slidable relative to the catheter and its linear position can be controlled by a user, e.g., via a pull wire that extends from the collar to the proximal end of the catheter. Thus, the collar can be translated to compress the coil to expand its radial shape or to stretch the coil to reduce its radial shape. The catheter further comprises side holes which may be used to deliver contrast, saline, or other media. The device may be placed in the same manner as a standard NG tube.

FIG. 5a shows a catheter device 230 including a catheter 232 with a coil 240 fixed thereto in an insertion state (first shape) within the patient anatomy 120 of FIG. 1b according to a third example of these exemplary embodiments; FIG. 5b shows the catheter device 230 of FIG. 5a with the coil 240 transitioned into an expanded state (second shape) by a collar 246 according to the third exemplary embodiment. Similar to the catheter devices 200, 212 of FIGS. 3-4, the catheter device 230 can be introduced into the esophagus, passed through the stomach past the stricture and placed such that a distal end 234 thereof is within the small bowel. The patient anatomy 120 shown in FIGS. 5a-b comprises the jejunum 136, similar to the preceding figures.

The catheter 232 extends from a proximal end (not shown) to a distal end 234 with an atraumatic distal tip. In this example, a first distal portion 236 of the catheter 214 includes multiple holes (not labeled) for providing an outlet for fluid introduced via the lumen of the catheter 232. In other examples, the first distal portion 236 comprises a single hole. In this example, the catheter device 230 further includes the coil 240 and the collar 246. A second distal portion 238 of the catheter 232 provides a surface over which the coil 240 is disposed and over which the collar 246 can translate. In this example, the second distal portion 238 is distal to the first distal portion 236 comprising the holes. In other embodiments, the second distal portion 238 over which the coil 240 and the collar 246 are disposed can be proximal to the first distal portion 236 or coincide wholly or in part with first distal portion 236.

A first end 242 of the coil 240 is fixed to the catheter 232 and a second end 244 of the coil 240 is fixed to the collar 246. As shown in FIG. 5a, when the collar 246 is in a first position (distal-most position), the coil 240 is stretched and assumes a first shape that is wrapped around the second distal portion 238 of the catheter 232 with minimal radial expansion relative to the catheter 232, e.g., the insertion state. Once placed, the collar 246 can be actuated to slide linearly in the proximal direction, e.g., to a second position (proximal-most position). The user pulls the collar 246 proximally thus forcing the coil 240 to be shortened and displaced, expanding radially outward from the surface of the catheter and assuming a second shape, e.g., the expanded state, as shown in FIG. 5b.

The coil 240 can expand to varying degrees based on the position of the collar 246 and the degree to which the collar 246 compresses the coil 240. To transition the coil 240 back into the first shape, the collar 246 can be advanced distally relative to the catheter 232. The collar 246 can be translated proximally and distally relative to the catheter 232 via any suitable mechanism, e.g., pull wire. It should be understood that the coil and the collar described above may be configured differently so that, e.g., distal advancement of the collar 246 compresses the coil 240. The coil 240 can be transitioned into the first state to withdraw the catheter device 230 from the patient anatomy.

The action of the coil 240 expanding into the second state expands the tissue of the surrounding jejunum 136 and anchors the catheter 232 in place. Similar to the catheter devices 200, 212 described above with regard to FIGS. 3-4, the catheter device 230 can be used for providing fluid for visualizing the small bowel for an EUS-GE procedure or can be placed further proximally for use as a feeding tube. In the EUS-GE applications, the catheter device 230 can provide the physician with a target that is visible under ultrasound.

In another aspect of these exemplary embodiments, a catheter device can include a wireform of various shapes deployable from a catheter to provide a radial expansion for maintaining a desired position of the catheter in the small bowel. In these embodiments, the catheter device includes a mechanism for fixing the wireform to the catheter. In some embodiments, the wireform can comprise an elongated proximal portion and a shaped distal portion, a proximal end of the wire remaining outside the proximal end of the catheter that can be fixed to the catheter when the wireform is advanced distally such that the shaped distal portion is located as a desired position in the small bowel. In some embodiments, the wire can be advanced through the interior of the catheter, e.g., via the primary lumen of the catheter. In other embodiments, the wire can be attached to an exterior of the catheter and deployed therefrom, similar to the catheter devices 200, 212 of FIGS. 3-4. Similar to the catheter devices 200, 212 of FIGS. 3-4, the catheter devices of the present embodiments can be one-time deployable or can be re-constrainable.

FIG. 6a shows a first wireform 250 according to a fourth example of these exemplary embodiments. The first wireform 250 comprises a Y-shape. FIG. 6b shows a second wireform 252 according to a fifth example of these exemplary embodiments. The second wireform 252 comprises a waveform. FIG. 6c shows a third wireform 254 according to a sixth example of these exemplary embodiments. The third wireform 254 comprises a C-shape. FIG. 6d shows a fourth wireform 256 according to a seventh example of these exemplary embodiments. The fourth wireform 256 comprises a mass of coiled up wire, e.g., similar to a coronary guidewire.

In some embodiments, the wireforms 250-256 can be passed through the lumen of the catheter (or through a second lumen of the catheter separate from the first lumen for administering the fluid). The interior of the catheter can constrain the wireforms 250-256 such that the wireforms 250-256 assume the first shape suitable for advancing the catheter to the target location. To transition the wireforms 250-256 into the second (expanded) shape, the wireforms 250-256 can be advanced distally so that the wireforms 250-256 extend out the (open) distal end of the catheter and expand under their natural bias to engage the inner walls of the small bowel. Once deployed, the wireforms 250-256 can be fixed to the catheter, e.g., at a proximal end by the operating physician, such that the catheter remains in fixed position with the wireforms 250-256 in the small bowel. In these embodiments, the wireforms 250-256 may be detached from the catheter and withdrawn proximally relative to the catheter to re-constrain the wireforms 250-256 into the first state and withdraw the catheter.

In other embodiments, the wireforms 250-256 can be fixed to the exterior of the catheter, similar to the catheter devices 200, 212 of FIGS. 3-4, and can be deployed similarly, e.g., via sheath/sleeve, pull wire, etc.

Similar to the preceding embodiments, the catheter devices comprising the wireforms 250-256 can be used for providing fluid for visualizing the small bowel for an EUS-GE procedure or can be placed further proximally for use as a feeding tube.

In another aspect of these exemplary embodiments, a catheter device can include Malecot style features. In particular, the catheter device can include a number of longitudinal wires arrayed circumferentially around a catheter that can transition between a first shape that is elongated or straight (insertion state) and a second shape that is curved radially outward from the catheter (expanded state), e.g., a bowed shape. In one example, 4 or more longitudinal wires can be arranged around a portion of the catheter such that, when the wires are expanded, an enlarged volume is defined that can contact the inner walls of the small bowel to enlarge the small bowel and/or anchor the catheter in place.

Those skilled in the art understand a Malecot catheter or Malecot anchor typically refers to catheters comprising longitudinal cuts forming arms/sections between adjacent cuts. For example, four longitudinal cuts of predetermined length may be spaced equally around the circumference of the catheter, thus forming four sections along this length. These sections remain straight during insertion and can then be deployed by pulling a distal portion of the catheter (e.g., distal to the Malecot sections) proximally (e.g., from a position proximal to Malecot sections) so that the Malecot sections are compressed and flair out, e.g., into “arms.” In one example, the Malecot sections can be deployed by pulling an internal catheter connected to the distal tip of an external catheter (the external catheter comprising the Malecot sections) proximally. The Malecot sections expand to anchor the catheter in place.

In the present embodiments, a variation of the Malecot includes forming the “arms” of the expanding section from separate wires instead of the arms being integral to the catheter. Wire arms have the advantage that they can provide the same strength with less mass. The wires of this device could be routed along multiple lumens arrayed circumferentially around the catheter.

In EUS-GE applications, the Malecot-style wires can be deployed, e.g., transitioned into the second state to expand the small bowel, so that fluid can be instilled to visualize the target location for placing the gastro-jejunal stent. In one example, a needle can be used to gain access to the jejunum in a space between adjacent arms. The arms may then be collapsed, e.g., transitioned into the first state, before introducing a guidewire. Alternatively, direct access to the small bowel can be gained (e.g., no guidewire) and the arms can be collapsed before deploying the stent. It is noted that placing the stent prior to collapsing the arms is not recommended as the stent could get caught in the arms. The wires may be coated with an insulating layer so that, if an electrically active stent delivery system is brought into close proximity to the arms at any time during the EUS-GE procedure, the arms will not electrically couple with the active electrode of the stent delivery system.

FIG. 7a shows a catheter device 260 including a catheter 262 with multiple longitudinal wires 268 arrayed circumferentially around a second distal portion 266 thereof in an insertion state (first shape) within the patient anatomy 120 of FIG. 1b according to an eighth example of these exemplary embodiments; FIG. 7b shows the catheter device 260 of FIG. 7a with the wires 268 transitioned into an expanded state (second shape) according to the eighth exemplary embodiment. Similar to the catheter devices of the preceding embodiments, the catheter device 260 can be introduced in the esophagus, passed through the stomach past the stricture and placed such that a distal end 264 thereof is within the small bowel. The patient anatomy 120 shown in FIGS. 7a-b comprises the jejunum 136, similar to the preceding figures.

The catheter 262 extends from a proximal end (not shown) to a distal end 264 with an atraumatic distal tip. A first distal portion (not labeled) of the catheter 262 can provide an outlet for fluid introduced via the lumen of the catheter 262. Similar to the preceding embodiments, the first distal portion can include one or more holes for discharging the fluid. In this example, the first distal portion coincides at least in part with a second distal portion 266 over which the wires 268 are arrayed, as shown in FIG. 7b, where saline/contrast 269 is discharged in the region adjacent to the wires 268. In other embodiments, the first distal portion comprising the fluid outlet can be provided proximally or distally to the second distal portion 266.

In this example, the catheter device 260 further includes the wires 268 arrayed circumferentially around the second distal portion 266. Each wire 268 can be fixed on its proximal end to the catheter and extend longitudinally along the surface or along channels in the wall of the second distal portion 266 to a distal end fixed to an inner member (not labeled) of the catheter device 260. For example, the inner member can be an inner catheter that can extend from a proximal end proximal to the catheter 262 to a distal end disposed at or near the distal end 264 of the catheter 262.

As shown in FIG. 7a, when the inner member is in a first position (distal-most position), the wires 268 are stretched longitudinally such that the wires 268 are substantially parallel with the catheter 262 with no or minimal radial expansion relative to the catheter 262, e.g., in the first shape or insertion state. Once the catheter 262 is placed at a desired location in the small bowel, the proximal end of the inner member can be pulled so that the distal ends of the wires 268 are drawn proximally thus forcing the wires 268 to bend radially outward and assume a second shape or expanded state, as shown in FIG. 7b. The expanded state of the wires 268 defines a volume that can enlarge the jejunum and/or anchor the catheter 262 in place.

As described above, in this example, an inner catheter is used to transition the wires 268 into the second shape. However, those skilled in the art will understand that other mechanisms can be used to translate the distal end of the wires 268 relative to the proximal ends. In some cases, it may be preferred for the inner member to be compressible, e.g., comprising an open spring or a gap. If a total gap is left between the distal and proximal ends the distal end can be kept in alignment with the proximal end by a preloaded guidewire. If a guidewire is used to align the ends of the inner member it would need to be linearly free to move and thus would not comprise the pulling element.

In one example, a further compressible member could be used to connect the proximal end to the distal end and a member could be connected to the distal end and controlled at the proximal control handle. By pulling on the distal end connected member from the proximal control handle the compressible section would shorten thus forcing the expansion of the outer wires. The guidewire can run through this lumen but would be free from the linear motion of the lumen. The lumen would be rigidly attached to the distal end but free to move with respect to the proximal end.

Similar to the catheter devices of the preceding embodiments, the catheter device 260 can be used for providing fluid for visualizing the small bowel for an EUS-GE procedure, e.g., the saline/contrast 269 shown in FIG. 7b, or can be placed further proximally for use as a feeding tube. In the EUS-GE applications, the catheter device 260 can provide the physician with a target that is visible under ultrasound. In feeding tube applications, another embodiment could be to compress the wires from a collar in the proximal end causing them to bow in the distal region where they are unconstrained by the catheter channels. This method could have some challenges with translating the compressive force along the wires but would allow a larger central lumen for feeding.

In another aspect of these exemplary embodiments, a catheter device comprises a spring placed over the catheter that is permanently attached to a distal portion of the catheter at one end. Once the catheter is positioned as desired, the spring can close and, in doing so, grasp tissue around the catheter to prevent the catheter from migrating. In some embodiments, this spring can be made of a shape memory alloy such that it returns to its original shape (closed spring) when heated. One example preset temperature could be that of the body, which would allow the spring to slowly move to its closed position as the catheter is fed through the body. The transition profile of the material could be designed such that the spring does not close until a sufficient time has elapsed to guide the tube to its target position.

FIG. 8a shows a catheter device 270 including a catheter 272 with a second distal portion 276 on which a spring 278 can be fixed according to a ninth example of these exemplary embodiments; FIG. 8b shows the spring 278 of the catheter device 270 of FIG. 8a in an insertion state (first shape) according to the ninth exemplary embodiment; FIG. 8c shows the spring 278 of FIG. 8b in an expanded state (second shape) according to the ninth exemplary embodiment. Similar to the catheter devices of the preceding embodiments, the catheter device 270 can be introduced in the esophagus, passed through the stomach past the stricture and placed such that a distal end 274 of the catheter 272 is within the small bowel.

The catheter 272 extends from a proximal end (not labeled) at a handle 279 of the catheter device 270 to a distal end 274 with an atraumatic distal tip, as shown in FIG. 8a. A first distal portion (not shown) of the catheter 272 is configured to provide an outlet for fluid introduced via the lumen of the catheter 272. Similar to the preceding embodiments, the first distal portion can include one or more holes for discharging the fluid and can coincide at least in part with a second distal portion 276 over which the spring 278 is disposed, as shown in FIGS. 8b-c, or the first distal portion can be provided proximal or distal to the second distal portion 276.

The second distal portion 276 provides a surface over which spring 278 is disposed. The spring 278 can be fixed to the catheter 272 at either its proximal or distal end, such that a remainder of the spring 278 is free to expand (open) or contract (close). As shown in FIG. 8b, when the spring 278 is in a first shape (insertion state), the spring 278 is elongated and open. As shown in FIG. 8c, when the spring 278 is in a second shape (expanded state), the spring 278 is compressed and closed.

As described above, the radial expansion and closure of the spring 278 within the small bowel can grasp the inner tissue of the small bowel to retain the catheter 272 in place. This can be achieved by using a shape memory alloy activated by heat. Similar to the catheter devices of the preceding embodiments, the catheter device 270 can be used for providing fluid for visualizing the small bowel for an EUS-GE procedure or can be placed further proximally for use as a feeding tube. In the EUS-GE applications, the catheter device 270 can provide the physician a target that is visible under ultrasound.

In a similar embodiment, a wire is attached alongside side of the tube which will later expand into a coil that pinches and anchors the tube to the jejunum wall. The wire alongside the tube ensures this anchoring mechanism maintains a low-profile during insertion and positioning of the tube in the patient. Once the tube is in place, the wire is activated to coil into a shape of larger diameter than the tube and with close enough pitch to clamp sections of the tissue wall (similar tissue getting caught within the rungs of a spring). As with the previous example, the wire could be shape memory or heat activated, leveraging the temperature of the body, or being activated by a different temperature which is applied by the physician or procedure support staff. If the temperature is deliberately activated, the wire itself could be activated or a core within the wire which heats and reshapes only the portion of the wire that it meant to coil. In another embodiment, the wire may not be heat activated but could include a core that, when pulled by the physician or procedure support staff, could reshape a section of the wire into a coil capable of clamping sections of the tissue wall.

In another aspect of these exemplary embodiments, a catheter device can include one or more flange-like features (referred to herein as a “flange” for ease of description). The flange may be formed in a similar manner as a stent, e.g., with a structure comprising interwoven or braided wires to form a wire mesh. In its natural (unconstrained) state, the stent can comprise a flared shape having a size (e.g., length cross-sectional diameter at its flared end) selected for placement within a body lumen. This structure can be compressible and/or constrainable for delivery to a target region using a catheter, at which time it can be expanded and/or unconstrained to transition into its natural size to engage the inner walls of the lumen.

In some embodiments, the catheter device comprises two re-constrainable braided/coated flanges. The catheter device incorporates three concentric members that allow it to deploy and re-constrain the two flanges. The flanges prevent migration bidirectionally and lock in fluid to create a visible pocket when fluid is administered. The flanges remain attached to the delivery device members and can be re-constrained at the end of the procedure for removal. Although FIGS. 9a-b described below show two flanges, those skilled in the art will ascertain that in other embodiments only a single flange can be used.

FIG. 9a shows a catheter device 280 including a catheter 282 with a first flange 294 (distal flange) and a second flange 298 (proximal flange) in an insertion state (first shape) according to a tenth example of these exemplary embodiments; FIG. 9b shows the catheter device 280 of FIG. 9a with the first flange 294 transitioned into an expanded state (second shape) according to the tenth exemplary embodiment; FIG. 9c shows the catheter device 280 of FIG. 9b with the second flange 298 transitioned into an expanded state (second shape) according to the tenth exemplary embodiment; FIG. 9d shows the catheter device 280 of FIG. 9c deployed in the patient anatomy 120 of FIG. 1b according to the tenth exemplary embodiment.

The catheter 282 extends from a proximal end (not shown) to a distal end 284 with an atraumatic distal tip. The catheter 282 includes a lumen 286 extending through the catheter 282 to an opening at the distal end 284. The lumen 286 of this embodiment serves two purposes, specifically, providing a path for fluid to be discharged and containing a first flange 294 and its corresponding first member 290 for deploying the first flange 294. The catheter 282 further includes a structure 288 extending radially outward from catheter 282 near its distal end 284 and turning proximally to form a circular pocket of predetermined depth for containing a second flange 298 and a distal portion of its corresponding second member 296 for deploying the second flange 298.

The first member 290 extends through the lumen 286 and is translatable relative to the catheter 282. The first member 290 includes a number of protrusions 292 (e.g., at least two) or one unitary ring extending radially outward near the distal end of the first member 290. A proximal end of the first flange 294 can be fixed to the protrusions 292 such that translating the first member 290 translates the proximal end of the first flange 294. As shown in FIG. 9a, the first flange 294 can be constrained within the lumen 286 of the catheter when the first member 290 is at a first position (proximal-most position), e.g., in a first (constrained) shape or insertion state. To transition the first flange 294 into the second (expanded) shape or expanded state, the first member 290 is advanced distally to push the first flange 294 out the distal end 284 of the catheter 282, as shown in FIG. 9b.

The second member 296 extends outside the catheter 282 and is translatable relative to the catheter 282. The second member 296 includes a number of protrusions 297 (e.g., at least two) or one unitary ring extending radially outward at the distal end of the second member 296. A distal end of the second flange 298 is, in this embodiment, fixed to the protrusions 297 such that translating the second member 296 translates the distal end of the second flange 298. As shown in FIGS. 9a-b, the second flange 298 is constrained within the structure 288 (e.g., pocket) extending radially off the catheter 282 when the second member 296 is at a first position (distal-most position), e.g., in a first (constrained) shape or insertion state. To transition the second flange 298 into the second (expanded) shape or expanded state, the second member 296 is withdrawn proximally to pull the second flange 298 out the proximal end of the structure 288, as shown in FIG. 9c.

Accordingly, the catheter device 280 is inserted with both flanges 294, 298 fully constrained between the concentric members of the catheter device 280, e.g., the first member 290 (innermost), the catheter 282 (middle) and the second member 296 (outermost). Once in position, the first flange 294 (distal flange) is deployed by advancing the first member 290. The second flange 298 (proximal flange) is then deployed by retracting the second member 296. As shown in FIG. 9d, the catheter device 280 is then anchored, e.g., within the jejunum 136.

As shown in FIG. 9c, fluid can be injected via the lumen 286 between the first member 290 and the inner wall of the catheter 282 out the distal end 284 of the catheter on the proximal side of the expanded first flange 294, thus creating a volume suitable as an EUS target. After use, the procedure can be reversed, the flanges re-constrained and the device removed.

A catheter device according to another exemplary embodiment includes a stiff, pre-formed superelastic wire that is fully deployed into the GI tract, e.g., pushed out of a catheter, and is shaped/deployed such that the wire draws the duodenal anatomy into a desired alignment. The ends of the device have a soft, atraumatic tip to prevent perforation. This can be achieved in multiple ways including coils, as shown below, soft polymer tip materials, sponges, etc.

FIG. 10a shows a catheter device 300 including a catheter 302 with a lumen 308 carrying a wire 310 in an insertion state (first shape) according to an eleventh example of these exemplary embodiments; FIG. 10b shows the catheter device 300 of FIG. 10a deployed in the patient anatomy 120 of FIG. 1b with a distal end 314 of the wire 310 pushed out a distal end 306 of the catheter 302 according to the eleventh exemplary embodiment; FIG. 10c shows the catheter device 300 of FIG. 10b deployed in the patient anatomy 120 of FIG. 1b with a medial portion 315 of the wire 310 pushed out the distal end 306 of the catheter according to the eleventh exemplary embodiment; FIG. 10d shows the wire 310 of the catheter device 300 of FIG. 10c in a deployed state (second shape) in the patient anatomy 120 of FIG. 1b according to the eleventh exemplary embodiment; FIG. 10e shows an alternative view of the wire 310 of the catheter device 300 of FIG. 10d in the deployed state (second shape) in the patient anatomy 120 of FIG. 1b according to the eleventh exemplary embodiment.

Similar to the preceding embodiments, the catheter device 300 is configured to be introduced into the esophagus, passed through the stomach past the stricture and placed such that a distal end 306 of the catheter 302 is within the small bowel. The patient anatomy 120 shown in FIGS. 10b-e comprises the jejunum 136, similar to the preceding figures, and FIGS. 10b-d further show the stomach 126.

As shown in FIG. 10a, the catheter 302 extends from a proximal end 304 to a distal end 306 with an atraumatic distal tip. In this example, the catheter 302 comprises a simple tube with a lumen 308. The lumen 308 contains the wire 310 and a distal portion of a pushing member 316. The pushing member 316 extends from a proximal end 318 (e.g., handle) to a distal end 320 that is configured to contact and push the wire 310 out the distal end 306 of the catheter 302.

The wire 310 extends from a proximal end 312 to a distal end 314. In this example, the proximal end 312 and the distal end 314 form coiled shapes when the wire 310 is unconstrained. A medial portion 315 of the wire 310 forms a curved shape when the wire 310 is unconstrained, the curve bringing the proximal end 312 and the distal end 314 into close contact. To be described below, the medial portion 315 of this embodiment is shaped based on a target anatomy, e.g., an approximate path from the stomach 126, through the pylorus and the duodenum to the jejunum 136.

The wire 310 is constrained within the lumen 308 of the catheter in a first shape (insertion state). To advance the wire 310 out the distal end 306 of the catheter 302 the wire 310 is pushed by the pushing member 316. More particularly, the catheter 302 is withdrawn through the patient anatomy 120 relative to the wire 310, which is configured to be deployed in its current position in the patient anatomy 120 as the catheter 302 is withdrawn. The catheter device 300 is, for example, extended under fluoroscopic guidance to the ligament of Treitz. As shown in FIG. 10b, withdrawing the catheter 302 proximally deploys the distal end 314 of the wire 310 in the jejunum 136. The distal end 314 of the wire 310 in this example coils upon release from the catheter 302. As shown in FIG. 10c, withdrawing the catheter 302 further proximally deploys the medial portion 315 of the wire 310 within the duodenum and the stomach. The medial portion 315 is configured, in this embodiment, to substantially conform to the shape of the patient anatomy 120 between the jejunum 136 and the stomach 126, e.g., C-shaped.

As shown in FIG. 10d, withdrawing the catheter 302 further proximally deploys the proximal end 312 of the wire 310 in the stomach. The fully released wire 310 pulls the duodenal anatomy into alignment with the gastric wall acts to draw the stomach and jejunum close together. As shown in FIG. 10e, in an EUS-GE procedure, the wire provides a visual target from an endoscopic view of a location for placement of a gastro-jejunal stent.

It may be noted by those knowledgeable in the art that any of the above embodiments may be combined in any manner not inconsistent with their operation and design to provide a system to enable ostomy management utilizing any or all of the characteristics of the various embodiments.

1-15. (canceled)

16. A device for administering a fluid to a patient anatomy, comprising:
a flexible catheter including a first lumen extending therethrough, the catheter being sized and shaped to extend to a target location within a gastrointestinal (GI) system, a first distal portion of the catheter including one or more exit ports communicating with the first lumen for administering a fluid to the target location; and
at least one wireform attached around the catheter or deployable from within the catheter, the wireform being formed so that, in a first state, the wireform assumes a first shape suitable for guiding the catheter to the target location and, in a second state, the wireform assumes a second shape in which the wire extends away from the catheter in at least one radial direction for contacting a wall of the target location to anchor the catheter in the target location while administering the fluid via the first lumen.

17. The device of claim 16, wherein the wireform is a stent comprising a wire mesh with a tubular structure, the stent assuming the second state under a natural bias and being collapsible into the first state around a second distal portion of the catheter, wherein the stent is releasable from the first state to transition into the second state by actuation proximal to the catheter.

18. The device of claim 17, further comprising:
a sheath disposed around the second distal portion of the catheter, the sheath being translatable relative to the catheter from a first position to a second position, wherein the stent is constrained by the sheath around the catheter in the first state when the sheath is in the first position and translating the sheath into the second position unconstrains the stent to allow the stent to assume the second state.

19. The device of claim 18, wherein the sheath can be translated from the second position to the first position so that the stent can be repositioned after initial deployment or so that the device can be withdrawn through the GI system.

20. The device of claim 16, wherein the wireform is a coil extending from a first end fixed to an outer surface of the catheter in a helical shape to a second end not fixed to the catheter, the coil assuming the first state when the second end is located away from the first end such that the coil is lengthened and the helical shape is compressed radially, the coil assuming the second state when the second end is drawn toward the first end such that the coil is shortened and the helical shape is expanded radially.

21. The device of claim 20, further comprising:
a collar disposed around a second distal portion of the catheter, the second end of the coil fixed to the collar, the collar being translatable relative to the catheter between a first position and a second position, wherein the coil is lengthened by the collar in the first state when the collar is in the first position and translating the collar into the second position provides a compressive force to the coil to transition into the second state.

22. The device of claim 16, wherein the wireform comprises a Y-shape, a waveform, a C-shape, or a coiled mass of wire.

23. The device of claim 16, wherein the wireform in the first state is translatable from a first position where the wireform is within and constrained by an interior of the catheter to a second position where the wireform extends out a distal end of the catheter and assumes the second state.

24. The device of claim 16, further comprising:
multiple wires, each wire extending longitudinally from a proximal end to a distal end, the distal end being translatable between a first position and a second position, the multiple wires being spaced circumferentially around the catheter,
wherein the wires in the first state are straight and translating the distal ends proximally causes the wires to bend radially outward relative to the catheter.

25. The device of claim 20, wherein the wireform is a spring attached to the catheter on a first end and not attached to the catheter on a second end, the spring assuming the first state when the second end is located away from the first end such that the coil is lengthened and the spring is compressed radially and spaces between rotations of the spring are open, the coil assuming the second state when the second end is drawn toward the first end such that the coil is shortened, the spring is expanded radially, and the spaces between the rotations of the spring are drawn close to grasp adjacent tissue.

26. The device of claim 16, further comprising:
at least one further wireform, the wireform comprising a distal anchor and the further wireform comprising a proximal anchor, fluid being instilled between the proximal and distal anchors to create a visible volume of fluid.

27. The device of claim 16, further comprising:
a first member extending through the first lumen of the catheter, wherein the wireform is a first flange fixed adjacent to a distal end of the first member, the first member being translatable between a first position wherein the first flange is within and constrained by the first lumen and a second position wherein the first flange extends out a distal end of the catheter and is allowed to assume the second shape to engage an inner wall of a small bowel.

28. The device of claim 27, further comprising:
a second member extending outside the catheter, the second member being translatable between a first position and a second position; and
a second flange fixed adjacent to a distal end of the second member,
the catheter including a pocket outside the catheter, wherein the second flange is within and constrained by the pocket when the second member is in the first position and the second flange extends out a proximal end of the pocket when the second member is in the second position so that the second flange is allowed to assume the second shape to engage the inner wall of the small bowel at a location proximal to that of the first flange.

29. The device of claim 28, wherein the fluid is instilled through the first lumen out the distal end of the catheter on a proximal side of the first flange so that a visible volume of fluid is created.

30. A device for providing a target location for an endoscopic ultrasound guided gastroenterostomy (EUS-GE) procedure, comprising:
a flexible catheter including a first lumen extending therethrough, the catheter being sized and shaped to extend to a target location within a gastrointestinal (GI) system;
a wire extending from a proximal end to a distal end that, in a first state, is disposed fully within the first lumen; and
a pushing element sized and shaped to extend through a proximal end of the first lumen so that a distal end of the pushing element can contact the proximal end of the wire to push the wire out a distal end of the catheter,
wherein, in a second state, the wire is pushed out the distal end of the catheter as the catheter is withdrawn through the GI system, the wire being formed so that, in the first state, the wire assumes a first shape constrained by the first lumen suitable for guiding the catheter to the target location and, in the second state, the wire assumes a second shape in which a medial portion of the wire curves to bring the proximal end and the distal end toward each other,
the wire being deployed in the GI system so that the distal end contacts a jejunal wall and the proximal end contacts a stomach wall to provide a target under visualization.

31. A method for administering a fluid to a patient anatomy, comprising:
guiding a flexible catheter to a target location within a gastrointestinal (GI) system, the catheter including a first lumen extending therethrough, a first distal portion of the catheter including one or more exit ports communicating with the first lumen for administering a fluid to the target location;
transitioning at least one wireform from a first state to a second state, the at least one wireform being attached around the catheter or deployable from within the catheter, the wireform being formed so that, in the first state, the wireform assumes a first shape suitable for guiding the catheter to the target location and, in the second state, the wireform assumes a second shape in which the wire extends away from the catheter in at least one radial direction for contacting a wall of the target location to anchor the catheter in the target location; and
administering the fluid via the first lumen.

32. The method of claim 31, wherein the wireform is a stent comprising a wire mesh with a tubular structure, the stent assuming the second state under a natural bias and being collapsible into the first state around a second distal portion of the catheter, wherein the stent is releasable from the first state to transition into the second state by a sheath disposed around the second distal portion of the catheter, the sheath being translatable relative to the catheter from a first position to a second position, wherein the stent is constrained by the sheath around the catheter in the first state when the sheath is in the first position and translating the sheath into the second position unconstrains the stent to allow the stent to assume the second state.

33. The method of claim 31, wherein the wireform is a coil extending from a first end fixed to an outer surface of the catheter in a helical shape to a second end not fixed to the catheter, the coil assuming the first state when the second end is located away from the first end such that the coil is lengthened and the helical shape is compressed radially, the coil assuming the second state when the second end is drawn toward the first end such that the coil is shortened and the helical shape is expanded radially via a collar disposed around a second distal portion of the catheter, the second end of the coil fixed to the collar, the collar being translatable relative to the catheter between a first position and a second position, wherein the coil is lengthened by the collar in the first state when the collar is in the first position and translating the collar into the second position provides a compressive force to the coil to transition into the second state.

34. The method of claim 31, wherein the wireform comprises a Y-shape, a waveform, a C-shape, or a coiled mass of wire, wherein the wireform in the first state is translatable from a first position where the wireform is within and constrained by an interior of the catheter to a second position where the wireform extends out a distal end of the catheter and assumes the second state.

35. The method of claim 31, wherein the wireform comprises multiple wires, each wire extending longitudinally from a proximal end to a distal end, the distal end being translatable between a first position and a second position, the multiple wires being spaced circumferentially around the catheter,
wherein the wires in the first state are straight and translating the distal ends proximally causes the wires to bend radially outward relative to the catheter.
